(19) United States
(12) Patent Application Publication
(10) Pub. No.: US 2020/0085554 A1
(43) Pub. Date: Mar. 19, 2020

(54) TEETH ILLUMINATION DEVICE WITH A LIGHT MIXING CHAMBER

(71) Applicant: KONINKLIJKE PHILIPS N.V., EINDHOVEN (NL)

(72) Inventors: OLAF THOMAS JOHAN ANTONIE VERMEULEN, OSS (NL); NIGEL DAVID YOUNG, MEADVALE (GB); PASCAL JEAN HENRI BLOEMEN, EINDHOVEN (NL)

(21) Appl. No.: 16/472,877

(22) PCT Filed: Dec. 28, 2017
(86) PCT No.: PCT/EP2017/084738

§ 371 Date: Jun. 23, 2019

(30) Foreign Application Priority Data
Dec. 30, 2016 (EP) 16207496.7

(51) Int. Cl. A61C 19/06; A61N 5/06

(52) U.S. Cl. CPC A61C 19/066; A61N 5/062; A61N 2005/0606; A61N 5/0603

(57) ABSTRACT

The invention relates to an oral care device for teeth whitening or other purpose. The device is designed to provide a uniform light intensity distribution at the teeth by use of a light mixing chamber of a mouthpiece comprised by the oral care device. The mouthpiece comprises a sealing structure with rims designed to connect to the gums of a user. At least apart of the rim and a closure of the mouthpiece surrounded by the rim are reflective. When light is emitted into the mouthpiece, the light will be reflected by the reflective rim, the reflective closure and the buccal side of the teeth. Accordingly, the light will be reflected multiple times, which makes the intensity distribution more uniform. Additionally, the mouthpiece may comprise light redirection structures arranged to redirect light rays from light injected into the mouthpiece.

### FIELD OF THE INVENTION

The invention relates to teeth illumination devices, particularly to oral hygiene devices such as teeth whitening devices.

### BACKGROUND OF THE INVENTION

Oral care devices such as teeth whitening devices use light for illuminating the teeth. The light can be used for different purposes including teeth whitening in combination with a whitening gel, plaque or bacteria hygiene by use of disinfecting UV light or other purposes. In order to be effective, the intensity of the light need to be sufficiently high. It may also be important that the light is distributed so that the light intensity is uniform over the teeth. In order to avoid damage of the pulp in the pulp chamber of the teeth it is also important that heating of the teeth is kept safely below a critical temperature.

Accordingly, there is a need for an oral care device which is capable of illuminating the teeth with a sufficiently high intensity, with a uniform intensity distribution, and which does not cause overheating of the teeth.

EP 1 649 827 A1 discloses a tooth whitening apparatus which comprises a tray for receiving in use an amount of a light-transmitting tooth whitening composition such that the tray may be positioned relative to a user's teeth such that at least a major portion of the exposed regions of the teeth may be immersed in use in said composition at one time. A light source is provided for introducing into the tooth whitening composition light to propagate around said tray and to be incident on the immersed surfaces of said tooth at least partially due to internal reflection at the interface of said tooth whitening composition with the internal tray surface.

The inventor of the present invention has appreciated that an improved oral care device is of benefit, and has in consequence devised the present invention.

### SUMMARY OF THE INVENTION

It would be advantageous to achieve an oral device capable of improving generation of a uniform light intensity at the teeth, alternatively or additionally capable of reducing heating of the teeth. In general, the invention preferably seeks to mitigate, alleviate or eliminate one or more of the above mentioned disadvantages singly or in any combination.

To better address one or more of these concerns, in a first aspect of the invention a mouthpiece for teeth illumination is presented that comprises:

an optically transparent body part, wherein the body part has a front side arranged to face a buccal side of the teeth of a user when the mouthpiece is placed in the mouth and an opposite back side,

a sealing structure comprising a rim circumscribing the body part, wherein the rim is shaped to contact gums of a user along upper and lower dental arches, and wherein the rim has a reflective surface arranged to reflect at least reflected light from the buccal side of the teeth,

one or more light sources arranged to transmit light into at least a part of the body part,

a transmission window located at the front side of the body part and arranged to transmit light from the body part towards the buccal side of the teeth and to transmit reflected light from the buccal side of the teeth into the body part, and

a back reflector arranged to reflect incident light from the body part, wherein the reflector extends over the back side of the body part, and wherein the buccal side of the teeth of the user together with the back reflector and the reflective surface of the rim forms a set of reflecting surfaces capable or reflecting light from the one or more light sources multiple times.

Advantageously, the mouthpiece is arranged to allow the light from the light sources to be reflected back and forth between the set of reflective surfaces. Accordingly, the amount of light power which is inappropriately absorbed by other surfaces than the surface of the teeth may be reduced and may lead to increased efficiency and thereby reduced heating of the teeth. Thus, light reflected by the teeth is recycled, which may lead to a higher irradiance. Furthermore, as the light may be reflected back and forth multiple times, the light from the light sources will be mixed which may lead to a more uniform light intensity distribution at the surface of the teeth.

According to an embodiment, the back reflector adjoins the reflective surface of the rim to form a single reflective surface facing the buccal side of the teeth when the mouthpiece is placed in the mouth. Since the back reflector and the reflective surface of the rim may be arranged adjacent to each other to form a connected set of two reflective surfaces the amount of light which escapes the mouthpiece may be reduced which may increase the efficiency.

According to an embodiment, the back reflector, the reflective surface of the part of the rim, and a buccal side of the teeth of the user together forms a closed or substantially closed set of reflective surfaces when the mouthpiece is placed in the mouth. Advantageously, by forming a closed or substantially closed reflective surface, the amount of lost light power may be reduced and the uniformity of the light intensity distribution may be improved.

According to an embodiment, the thermal conductivity of the back reflector is higher than the thermal conductivity of the body part to enable transport of heat from the one or more light sources. By configuring the mouthpiece so that the thermal conductivity of the back reflector is relatively high, waste heat from the light sources may primarily be transferred away by the back reflector so that the percentage of the waste heat which heats the teeth may be reduced. The improvement of heat transport away from the teeth may enable use of light sources having a higher light power.

According to an embodiment, the body part comprises a light redirecting structure arranged to change the propagation direction of light, wherein at least a part of the light redirecting structure is located between the one or more light sources and the transmission window. Advantageously, by changing the propagation direction of light rays from the light sources, unobstructed transmission of at least some of the light rays from the light sources are prevented which may lead to increased travel length of the light rays and thereby improved light mixing. Furthermore, the light redirection structure may make the intensity distribution less sensitive to distance variations of the placement of the mouthpiece relative to the teeth. The change of propagation direction may be achieve by configuring the light redirecting structure as a refracting light redirecting structure and/or as a reflecting light redirecting structure.

According to an embodiment, the light redirecting structure comprises one more refractive air cavities located between the one or more light sources and the transmission window. Advantageously, the refractive redirection structures may be formed as air cavities to achieve a simple production method. For example, the air cavities may be formed in the body part by forming air cavities, e.g. at an interface or boundary of the body part.

According to an embodiment, the one or more refractive air cavities are asymmetrically shaped so that the light from the light sources will be spread more in a direction parallel to a occlusal plane of teeth than a vertical plane perpendicular to the occlusal plane. Advantageously, asymmetrical spreading may improve the uniformity of the intensity distribution both in the occlusal plane and the vertical plane.

According to an embodiment, the light redirecting structure is opaque or translucent and arranged to redirect light through scattering and/or reflection. For example, an opaque structure having a scattering or reflective surface may be provided to redirect light, primarily through scattering or reflection. The scattered light will be reflected by the reflective surfaces and may improve creation of a uniform light intensity distribution. As an alternative to an opaque structure, the redirecting structure may be translucent, e.g. provided as a semi-transparent structure having light scattering properties, e.g. achieved by a transparent structure comprising embedded scattering particles.

According to an embodiment, the one or more light sources are arranged to emit light towards the light redirecting structure. Accordingly, at least some of the light rays from the light sources may be redirected by the redirecting structure to improve the distribution of light in the body part.

According to an embodiment, the sealing structure is arranged for retaining an applied dental substance within a space defined by the front side of the optically transparent body part, the rim and the buccal side of the teeth. Advantageously, the mouthpiece may comprise a sealing structure to help keeping the dental substance in place between the teeth and the transparent window of the mouthpiece. For example, the mouthpiece may be a teeth whitening mouthpiece and the dental substance may be a teeth whitening gel.

According to an embodiment, the one or more light sources are arranged between the transmission window and the back reflector. Advantageously, the light sources are not located at the transmission window, but at a distance from the transmission window to enable spreading of light before the light is transmitted through the transmission window and/or to enable placement of light redirection structures between the light sources and the transmission window.

According to an embodiment the one or more light sources are arranged to transmit light into the body part through holes in the back reflector. By arranging the light sources in this way, at least a part of the light source such as the part with electrical connection terminals may be arranged behind the reflector (seen from the buccal side of the teeth). This may be an advantage for the purpose enabling transport of heat from the light sources away from the teeth and/or for enabling simple electrical connection with the electrical connection terminals.

According to an embodiment, the body part comprises an outwardly protruding bite part shaped to form a bite surface, wherein the bite part comprises a reflective portion arranged to reflect incident light from the body part. The bite part may advantageously improve fitting of the mouthpiece to different mouth cavities. Advantageously, the reflective portion may prohibit loss of light into the bite part.

According to an embodiment, the body part comprises a curved surface where the back reflector extends over the curved surface of the back side and thereby forms a concavely shaped back reflector. A concavely shaped back reflector may be advantageous for the purpose of forming a reflector which provides a uniform distance between the reflector and the buccal side of the teeth for achieving a uniform light intensity distribution along the dental arch of the teeth.

A second aspect of the invention relates to use of a mouthpiece according to the first aspect for teeth illumination comprising the steps of

arranging the mouthpiece so that the rim contacts the gums along the upper and lower dental arches of a user and so that the buccal side of the teeth together with the back reflector and the reflective surface of the rim forms a set of reflecting surfaces capable or reflecting light from the one or more light sources multiple times, and

illuminating the teeth by light emitted through the transmission window so that a portion of the emitted light is reflected by the teeth back into the optically transparent body part to achieve multiple reflections via the back reflector and the reflective surface of the rim.

The use of the mouthpiece may include a step of applying a dental substance, e.g. teeth whitening gel, at the teeth, at the rim, the transmission window or a combination thereof.

In general the various aspects of the invention may be combined and coupled in any way possible within the scope of the invention. These and other aspects, features and/or advantages of the invention will be apparent from and elucidated with reference to the embodiments described hereinafter.

In summary the invention relates to an oral care device for teeth whitening or other purpose. The device may be designed to provide a uniform light intensity distribution at the teeth by use of a light mixing chamber of a mouthpiece comprised by the oral care device. The mouthpiece may comprise a sealing structure with a rim designed to connect to the gums of a user. At least a part of the rim and a closure of the mouthpiece surrounded by the rim are reflective. When light is emitted into the mouthpiece, the light will be reflected by the reflective rim, the reflective closure and the buccal side of the teeth. Accordingly, the light may be reflected multiple times which may provide a more uniform intensity distribution at increased irradiance. Additionally, the mouthpiece may comprise light redirection structures arranged to redirect light rays from light injected into the mouthpiece.

### BRIEF DESCRIPTION OF THE DRAWINGS

Embodiments of the invention will be described, by way of example only, with reference to the drawings, in which

FIG. 1 illustrates a mouthpiece for use as a teeth illumination device,

FIGS. 2A, 2B and 3 illustrate cross-sectional views of alternative embodiments of mouthpieces,

FIGS. 4A and 4B illustrate examples of the light redirection structure in cross-sectional views of the mouthpiece, and

FIG. 5 illustrate an example of the mouthpiece where the light redirection structure comprises refractive air cavities.

### DETAILED DESCRIPTION OF THE EMBODIMENTS

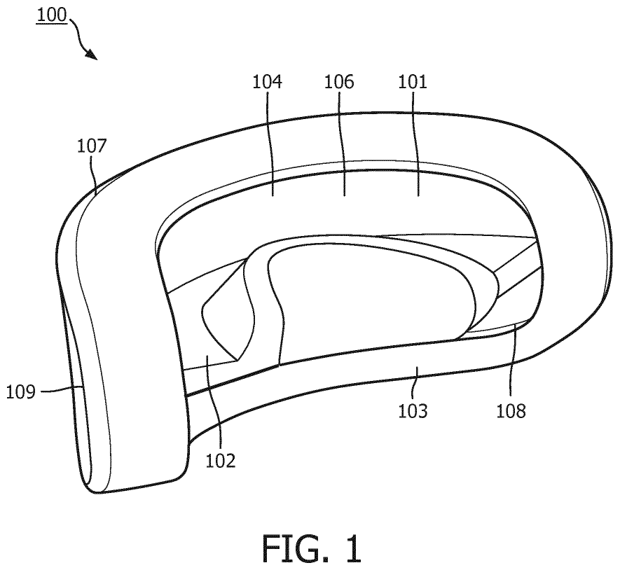

FIG. 1 illustrates a mouthpiece 100 for use as a teeth illumination device. The teeth of a user may be illuminated for the purpose of teeth whitening, plaque or bacteria hygiene, or other oral hygiene uses. The mouthpiece may be a consumer product for home use or a professional product to be used by a professional, in both cases the user, i.e. the home user or the patient, is the person that receives the illumination from the device.

The mouthpiece 100 comprises an optically transparent body part 101 having a front side 104 arranged to face a buccal side of the teeth of the user and a rim 103 which as illustrated may be formed as a closed rim which circumscribes the front side 104. Optionally, the the mouthpiece 100 may include a bite part 102 protruding from the front side 104. In case the mouthpiece 100 includes a bite part 102, the rim 103 may be formed so that it circumscribes the front side 104 with exception of opposite locations of the rim where the bite part 102 intersects the rim 103. At least a part of the front side 104 constitutes a transmission window 106 arranged to transmit light from the body part 101 towards the buccal side of the teeth and to transmit reflected light from the buccal side of the teeth back into the body part 101.

The bite part extends outwardly to form a bite surface, i.e. the illustrated plane of the bite part 102. The user can bite onto the upper and lower bite surfaces, i.e. so that the upper and lower occlusal sides of the teeth contact the bite surfaces. The bite part 102 enables the user to hold the mouthpiece 100 in place in the mouth and, thereby, provides better fixture of the mouthpiece. Additionally, the bite part 102 may improve the capability of the mouthpiece 100 to retain its shape when a force is applied to the mouthpiece 100, such as when the user applies a pressure to the bite part 102 so that rim makes contact with the gums. This is necessary in order to confine an applied dental substance, such as a teeth whitening gel, within the rim-structure.

The rim 103 may be shaped to contact gums of a user along the upper and lower dental arches. The rim 103 may have a reflective surface 108 which is provided to reflect back-reflected light from the teeth, particularly the buccal side of the teeth. A function of the rim is to serve as a sealing structure for preventing teeth whitening gel or other dental substances from leaking out and to assist in retaining the dental substance at a location of the teeth.

The mouthpiece 100 further comprises a back reflector 107 arranged to reflect incident light from the body part 101. The reflector extends over a back side 109 of the body part 101. The back reflector 107 together with the reflective rim surface 108 and the buccal side of the teeth forms a set of reflecting surfaces. As described below, the mouthpiece can be configured so that the set of reflecting surfaces are capable or reflecting light from light sources multiple times for mixing the light to achieve a uniform illumination of the buccal side of the teeth without intensity hotspots.

The body part 101 may be an optically transparent body part, i.e. transparent in the sense the light is not scattered. However, in practice the transparent material of the body part 101 may contain irregularities, e.g. particles, implying that some light scattering will take place. These particles may have been added purposely to increase illumination uniformity. Accordingly, the body part may be referred to as an optically translucent body part.

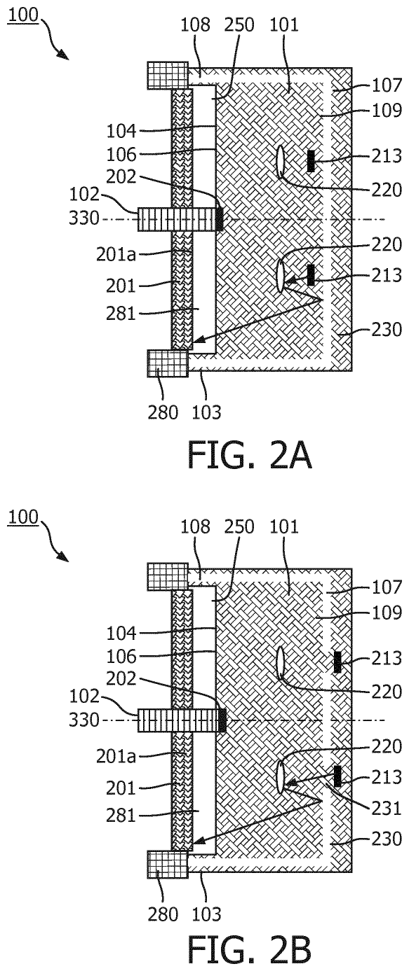

FIG. 2A shows a principal sketch of a cross-sectional view of the mouthpiece 100 in a plane perpendicular to the bite surface of the bite part 102, i.e. in a plane perpendicular to the occlusal plane 330. FIG. 2A shows the front side 104 of the body part 101, back side 109 of the body part 101, and the transmission window 106 of the front side 104. The front side 104 faces the buccal side 201a of the teeth 201. The rim 103 contacts the gums 280 at the upper and lower dental arches.

FIG. 2A shows two light sources 213 in the cross-sectional plane. In general the mouthpiece 100 may comprise one or more light sources 213 arranged to transmit light into at least a part of the body part 101.

A space 281 is defined between the front side 104 of the body part 101, the buccal side 201a, the rims 103 and the gums of the user 280. The space 281 may be used to contain a dental substance, e.g. a teeth whitening gel. The dental substance may have been applied by the user at the buccal side of the teeth or at the front side 104 of the mouthpiece body part before the mouthpiece is inserted. The rim 103 may be part of a sealing structure 250. Additionally, the optional bite part 102 may also be part of the sealing structure 250 since the bite part 102 restrains the location of the dental substance to above or below the occlusal plane 330.

FIG. 2A shows that the back reflector 107 is connected with the reflective surface 108 of the rim 103. Thus, the mouthpiece 100 may be configured so that the back reflector 107 adjoins the reflective surface 108 of the rim 103 to form a single reflective surface which faces the buccal side 201a of the teeth when the mouthpiece is placed in the mouth. Accordingly, the back reflector 107, the reflective surface 108 of at least a part of the rim 103, the buccal side 201a of the teeth form a closed or substantially closed set of reflective surfaces when the mouthpiece is placed in the mouth. Accordingly, light from the light sources 213 will be reflected back and forth multiple times which will improve the uniformity of the intensity distribution of the light at the teeth 201. It is clear that the reflective surfaces of the mouthpiece 100 and the teeth 201 may not form a completely closed reflective surface. For example, due to a portion of the gums 280 between the teeth 201 and the reflective surface 208 of the rim, or because the rim 103 may not perfectly connect with the gums at all locations, a small portion of the light may not be reflected but may be absorbed or escape. Accordingly, a substantially closed reflective surface may be created.

The back reflector 107 and the reflective surface 108 may be made by applying a diffuse reflective or specular reflective material, such as white paint or another substance, to the back side 109 of the body part 101 and to the rims 103, e.g. a part of the rims such as the surface intended to face the oral cavity. In another example, an opaque part 230 of the mouthpiece 100 comprises the back reflector 107, the rim 103 and the reflective surface 108. The opaque part 230 is connected with the back side 109 and possibly other portions of the body part 101 so that the opaque part 230 forms the rim 103, the reflective surface 108 and the back reflector 107 at the interfacing connection between the body part 101 and the opaque part.

As illustrated in FIG. 2A, the mixing of the light from the light sources 213 may be further improved by configuring the mouthpiece 100 with a light redirecting structure 220 arranged to change the propagation direction of the light from the one or more light sources 213. The light redirecting structure 220 may be configured in various ways, but so that at least a part of the light redirecting structure 220 is located between the one or more light sources 213 and the transmission window 106. As illustrated in FIG. 2A, the redirecting structure 220 is opaque and has a specular reflective or diffuse reflective surface so that the light from one of the light sources 213 is redirected through specular reflection or scattering. The light redirecting structure 220 could also be semi-transparent and have scattering properties, e.g. embodied by a transparent material filled with scattering particles. As another example, the light redirecting structure 220 could be transparent and arranged to redirect light through refraction. Thus, in general the one or more light sources 213 are arranged relative to the light redirecting structure 220 so that at least some of the emitted light from a light source 213 is redirected by an oppositely located redirection structure 220. Depending on at least the extension of the redirecting structure and the spreading angle of the light sources 213, it is possible that not all light from a light source is redirected, but preferably a substantial portion of the light should hit the redirection structure 220. In effect, the light redirection structure 220 forces light rays from a light source 213 to travel a longer distance before they hit the buccal side of the teeth, and as a consequence of this, the light intensity distribution at the teeth surface will be more uniform and less sensitive to distance variations so that the intensity distribution on offset teeth is largely maintained and/or so that the intensity on the teeth becomes less sensitive to distance variations between the teeth and the transmission window 106 due to natural variations in users oral cavity shapes and variations in the placement of the mouthpiece.

As illustrated in FIG. 2A the one or more light sources 213 are arranged between the transmission window 106 and the back reflector 107. Additionally, the one or more light sources 213 are arranged so that output apertures of the lights sources are distanced from the transmission window. Accordingly, in an embodiment without a light redirection structure 220, the light will at least have to travel a distance through the body part 101 before the light is transmitted through the transmission window 106.

FIG. 2A shows the outwardly protruding bite part 102. In order to avoid that light from the body part 101 is transmitted into the bite part 102 and thereby lost, the bite part may comprise a reflective portion 202 which reflect light back into the body part 101. The bite part 102 may be made from a different material than the body part 101. For example, the bite part may be made from an opaque material, e.g. a white material, so that the white portion of the bite part 102 that makes contact with the body portion 101 is the reflective portion 202. In another example, the body part 101 is made from transparent silicone and the bite part 102 is made from silicone filled with scattering particles like TiO2, AlO2 or other similar materials. Due to the scattering particles, the bite part 102 becomes substantially opaque and attains scattering properties similar to the reflective portion 202.

According to an embodiment the back reflector 107 and possibly the reflective surface 108 of the rims may have a high thermal conductivity, e.g. so that the thermal conductivity of the back reflector 107 is higher than the thermal conductivity of the body part 101. The relatively high thermal conductivity may be obtained by use of a paint for the reflectors 107, 108 which have good thermal conduction properties or by use of an opaque part 230 has good thermal conduction properties. For example, the opaque part 230 may be made from transparent silicone or other transparent material filled with particles which provides both scattering and heat conductive effects, e.g. particles like TiO2, AlO2 or other. The heat conductive property of the back reflector 107 helps transporting heat away from the one or more light sources 213 and thereby limits heating of the teeth 201. Heating of the teeth 201 may damage the pulp in the pulp chamber of the teeth. The one or more light sources 213 such as LEDs may be mounted on a printed circuit board so that heat from the light sources is mainly transported with the electrical connections of the light sources via the printed circuit board to the back reflector 107.

FIG. 2B shows an alternative configuration of the mouthpiece 100 where the one or more light sources 213 are arranged to transmit light into the body part 101 through holes 231 in the back reflector 107. For example, the one or more light sources 213 may be arranged behind the back reflector 107 when seen from the transparent window 106. Alternatively, the one or more light sources 213 may be arranged behind the holes 231 so that at least a part of a light source 213 extends into or through the hole. For example, the one or more light sources 213 may be arranged on a printed circuit board located behind the back reflector 107 so that the light sources protrudes from the printed circuit board towards the back reflector 107and at least partially through the holes 231.

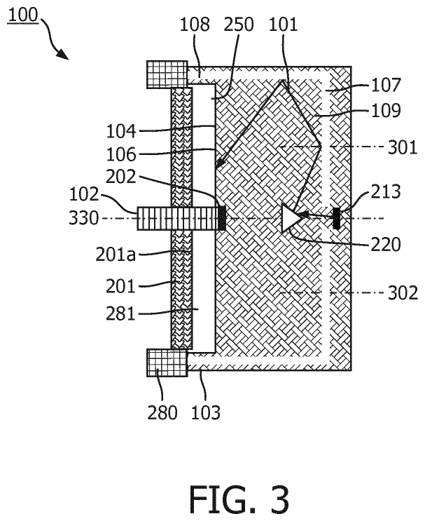

FIG. 3 shows another alternative configuration of the mouth piece 100 where the one or more light sources 213 is arranged in a single plane e.g. the occlusal plane 330. For example, a plurality of light sources may be arranged along a line extending in the occlusal plane 330 along the back side 109. In order to distribute the light from the single light source 213 or the single row of light sources 213, the light redirection structure 220 is configured to split the light from the light source 213 into a least two principal directions. The light redirection structure 220 may have specular reflective surfaces and could have a triangular shape or other shape consisting of a plurality of planar or curved reflective surface. The configuration of FIG. 3 with a beam splitting structure 220 could also be applied when the mouthpiece 100 is configured with a plurality of light sources arranged in a plurality of planes, e.g. along two rows of one or more light sources 213 in an upper plane 301 and a lower plane 302 being parallel with the occlusal plane 330.

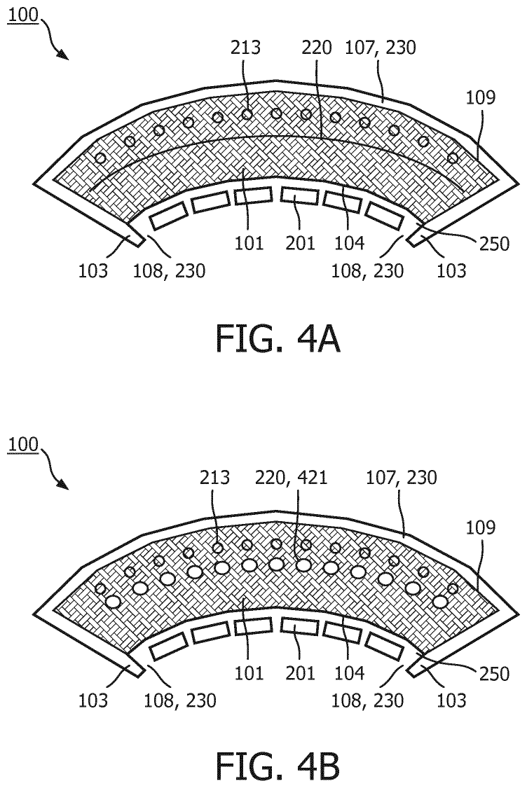

FIG. 4A shows a principal sketch of a cross-sectional view of an embodiment of the mouthpiece 100 along the occlusal plane 330. The optional bite part 102 is not included in this illustration. FIG. 4A shows the dental arch of teeth 201 and a curved body part 101 of the mouthpiece 100. A plurality of light sources 213 is arranged along a line in the curved body part. The optional light redirection structure 220 is in the form of a reflective unbroken strip extending along the same plane as the light sources 213. The back reflector 107 constitutes at least a part of the opaque part 230 connected with the back side 109 of the body part 101 and connected to the ends of the body part 101 so that the opaque part 230 forms the rim 103, the reflective surface 108 of the rim and the back reflector 107.

As shown in FIG. 4A, the back side 109 of the body part 101 may have a curved surface, e.g. which is curved to achieve a substantial constant perpendicular distance between the back side 109 and the teeth 201. In this case the back reflector 107 may extend over the curved surface of the back side 109 to form a concavely shaped back reflector 107. The curved back reflector 107 may be formed according to the different examples of the back reflector 107.

FIG. 4B shows a cross-sectional view of another embodiment of the mouthpiece 100 where the light redirection structure 220 comprises one or more refractive air cavities 421 located between the one or more light sources and the transmission window. As illustrated, one refractive air cavity 421 may be placed in front (when seen from the front side 104) of each light source 213. Alternatively, a single refractive air cavity 421 may be shaped so that it has an extension which covers two or more light sources, i.e. so that the single refractive air cavity 421 is shaped to redirect light from two or more light sources 213. The refractive air cavities 421 spread the light of the light sources 213 to create a uniform illumination distribution. The refractive air cavities 421 may be shaped as parabolic air cavities or may be shaped differently. Furthermore, the air cavities 421 may be asymmetrically shaped so that the light from the light sources 213 will be spread more in a direction parallel to the occlusal plane 330 of teeth (the plane of the cross-sectional view) than a vertical plane perpendicular to the occlusal plane or vice versa.

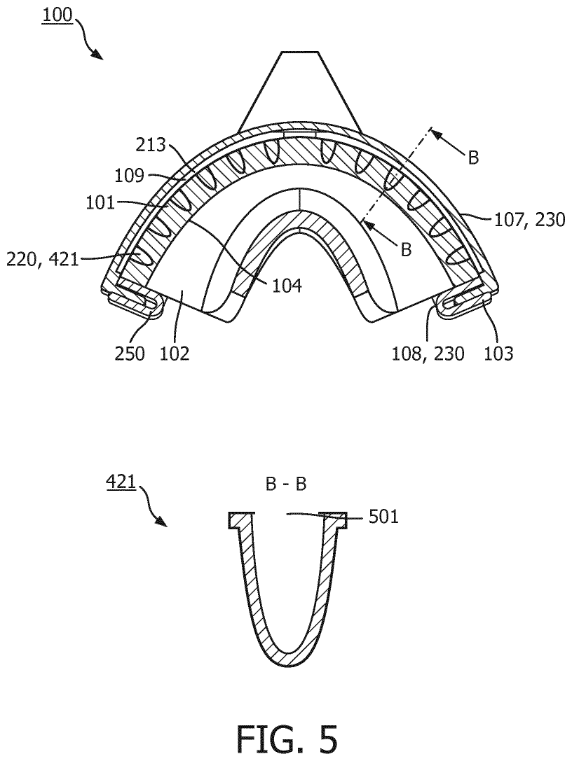

FIG. 5 shows a cross-sectional view of an embodiment of the mouthpiece 100 corresponding to the embodiment in FIG. 4B where light redirection structure 220 is constituted by refractive air cavities 421. The light sources 213 are not visible in the illustration, but may be located as indicated by reference 213 in front of the air cavities 221. For example, the light sources 213 may be located on a printed circuit board connected to the back side 109 of body part 101. The back reflector 107 is in the form of an opaque part 230 which also forms the rim 103 and the reflective surface 108 of the rim. The cross-sectional shape of one of the air cavities 421 is shown in section B-B. Accordingly, the air cavity 421 has a light receiving opening 501 which is located in front of a light source 213 so that substantially all light from the light source 213 is refracted at the interface between the air cavity and the surrounding material.

The air cavities 421 may be molded into the body part 101 so that the openings 501 are placed at the back surface 109 and thereby forms corresponding opening at the back surface 109. As noted, the light sources 213 can be placed behind the openings (when seen from the front surface 104) e.g. by use of a printed circuit board connected to the back surface 109. Accordingly, the light source 213 does not need to be embedded in the body part 101, but can be located on a separate part of the mouthpiece 100.

As an alternative to molding the air cavities 421 in the body part 101, the air cavities 421 can be created using separate parts, e.g. made from polymethl methacrylate (PMMA), which are subsequently mounted in front of the light sources 213.

As an alternative to integrating the one or more light sources 213 with the mouthpiece 100, light may be guided to the body part 101 or the light redirection structure 220 via light guides such as optical fibers. Accordingly, one or more external light sources may transmit light to the mouthpiece 100 via light guides as an alternative to using one or more light sources 213 integrated with the mouth piece 100.

The one or more integrated light sources 213 or external light sources may emit light within a range of wavelengths suitable obtaining a given result of the oral care. For example, light sources which emit light in an ultra violet range may be used for plaque or bacteria hygiene. For the purpose of teeth whitening, light sources which emit visible blue light may used.

The optically transparent body part 101 may be made from an optically transparent material such as silicone. The opaque part 230 could also be made from the same material as the optically transparent body part 101, e.g. silicone filled with scattering particles like TiO2, or other material. Preferably, the optically transparent body part 101, and/or optionally the opaque part 230 may be made from an elastic material, e.g. silicone, so that the mouthpiece 100 can be shaped to fit individual users and provide comfort for the user.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage.

Any reference signs in the claims should not be construed as limiting the scope.

1. A mouthpiece for teeth illumination, the mouthpiece comprising
an optically transparent body part, wherein the body part has a front side arranged to face a buccal side of the teeth of a user when the mouthpiece is placed in the mouth, and an opposite back side,
a sealing structure comprising a rim circumscribing the body part, wherein the rim is shaped to contact gums of a user along upper and lower dental arches, and wherein the rim has a reflective surface arranged to reflect at least a portion of the reflected light from the buccal side of the teeth,
one or more light sources arranged to transmit light into at least a portion of the body part,
a transmission window located at the front side of the body part and arranged to transmit light from the body part towards the buccal side of the teeth and to transmit reflected light from the buccal side of the teeth into the body part, and
a back reflector arranged to reflect incident light from the body part, wherein the reflector extends over at least a portion of the back side of the body part, and wherein the buccal side of the teeth of the user together with the back reflector and the reflective surface of the rim form a set of reflecting surfaces capable of reflecting light from the one or more light sources multiple times.

2. A mouthpiece according to claim 1, wherein the back reflector adjoins the reflective surface of the rim to form a single reflective surface facing the buccal side of the teeth when the mouthpiece is placed in the mouth.

3. A mouthpiece according to claim 1, wherein the back reflector the reflective surface of the rim, and the buccal side of the teeth of the user together forms a closed or substantially closed set of reflective surfaces when the mouthpiece is placed in the mouth.

4. A mouthpiece according to claim 1, wherein the thermal conductivity of the back reflector is higher than the thermal conductivity of the body part to enable transport of heat from the one or more light sources.

5. A mouthpiece according to claim 1, wherein the body part comprises a light redirecting structure arranged to change the propagation direction of light, wherein at least a portion of the light redirecting structure is located between the one or more light sources and the transmission window.

6. A mouthpiece according to claim 5, wherein the light redirecting structure is arranged to redirect light through refraction.

7. A mouthpiece according to claim 5, wherein the light redirecting structure comprises one or more refractive air cavities located between the one or more light sources and the transmission window.

8. A mouthpiece according to claim 7, wherein the one or more refractive air cavities are asymmetrically shaped so that the light from the light sources will be spread more in a direction parallel to an occlusal plane of teeth than a vertical plane perpendicular to the occlusal plane.

9. A mouthpiece according to claim 5, wherein the light redirecting structure is opaque or translucent and arranged to redirect light through scattering and or reflection.

10. A mouthpiece according to claim 5, wherein the one or more light sources are arranged to emit light towards the light redirecting structure.

11. A mouthpiece according to claim 1, wherein the sealing structure is arranged for retaining an applied dental substance within a space defined by the front side of the optically transparent body part, the rim and the buccal side of the teeth.

12. A mouthpiece according to claim 1, wherein the one or more light sources are arranged between the transmission window and the back reflector.

13. A mouthpiece according to claim 1, wherein the one or more light sources are arranged to transmit light into the body part through holes in the back reflector.

14. A mouthpiece according to claim 1, wherein the body part comprises an outwardly protruding bite part shaped to form a bite surface, wherein the bite part comprises a reflective portion arranged to reflect incident light from the body part.

15. A mouthpiece according to claim 1, wherein the back side of the body part comprises a curved surface, and wherein the back reflector extends over the curved surface of the back side and thereby forms a concavely shaped back reflector.

16. Use of a mouthpiece according to claim 1 for teeth illumination comprising the steps of
arranging the mouthpiece so that the rim contacts the gums along upper and lower dental arches of the user and so that the buccal side of the teeth together with the back reflector and the reflective surface of the rim forms a set of reflecting surfaces capable or reflecting light from the one or more light sources multiple times, and
illuminating the teeth by light emitted through the transmission window so that a portion of the emitted light is reflected by the teeth back into the optically transparent body part to achieve multiple reflections via the back reflector and the reflective surface of the rim.
